(19) United States
(12) Patent Application Publication
(10) Pub. No.: US 2016/0271096 A1
(43) Pub. Date: Sep. 22, 2016

(54) THERAPEUTIC OXIDATIVE FORMULATIONS AND METHODS OF USE THEREOF

(71) Applicant: OGENX THERAPEUTICS CORPORATION, Clearwater, FL (US)

(72) Inventors: Richard Davis, Clearwater, FL (US); Dmytro Berezhnoy, Savannah, GA (US)

(21) Appl. No.: 15/031,998

(22) PCT Filed: Oct. 23, 2014
(86) PCT No.: PCT/US14/62062

§ 371 Date: Apr. 25, 2016

Related U.S. Application Data
(60) Provisional application No. 61/895,590, filed on Oct. 25, 2013.
(60) Provisional application No. 61/895,570, filed on Oct. 25, 2013.

(51) Int. Cl. A61K 31/327; A61K 9/00; C07C 407/00; A61K 47/20

(52) U.S. Cl. CPC A61K 31/327; A61K 47/20; A61K 9/0019; C07C 407/00

(57) ABSTRACT

Disclosed are pharmaceutical compositions of multi-component systems which comprise at least one peroxidic species, solubilized with a stabilizing solvent, in combination with a chelating dye, and an aromatic redox compound. Further, the present invention relates to methods or use of the pharmaceutical composition for treating, preventing, or managing a disease of a subject in need thereof.

### CROSS-REFERENCE TO RELATED APPLICATION

The present application claims priority to U.S. Provisional Patent Application 61/895,570, filed 25 Oct. 2013, titled: Compositions, Methods of Use, and Mechanisms of Action of Therapeutic Oxidative Formulations; and 61/895,590, filed 25 Oct. 2013, titled: Pharmaceutical Preparation, Its Formulation, Synthesis, Method and Use for Restoring Retinal Architecture to Improve Vision in Age-Related Eye Disease, the entire contents of which are incorporated herein by reference in their entirety for all purposes.

### TECHNICAL FIELD

The present invention relates to pharmaceutical compositions of multi-component systems which comprise at least one peroxidic species, solubilized with a stabilizing solvent, in combination with a chelating dye, and an aromatic redox compound. Further, the present invention relates to methods of using the pharmaceutical composition for treating, preventing, or managing a disease of a subject in need thereof.

### BACKGROUND

Mitochondria have long been recognized as the “power house” of the cell because of their central role in the generation of adenosine triphosphate (ATP), the major source of energy used to power all metabolic functions in eukaryotic cells. The generation of ATP is conducted through a complex series of oxidative/reduction (redox) reactions. More recently mitochondria have been recognized as important mediators in the management of the oxidative state of the cell through management of various reactive oxygen species (ROS) generated in the cell through a variety of external and internal mechanisms. Redox imbalance though defects in the tightly controlled redox reactions within the mitochondria have been cited as playing multiple roles in the initiation, promotion and or maintenance of what is termed “oxidative stress”—now widely recognized to play a central role in the etiology of a wide range of diseases, especially chronic diseases, such as inflammatory diseases and cancers. Although the underlying chemistry was not appreciated, throughout history foods with antioxidant properties have been used to successfully treat many different types of diseases that are causally related to oxidative stress. With the emerging scientific understanding of the role of oxidative stress in disease, efforts by the pharmaceutical industry to identify pharmaceutical agents that could reset the redox state of a disease to a normal healthy state has begun to intensify.

Age-related acquired vision loss is principally associated with four common eye diseases—which include: a) Age-related Macular Degeneration (AMD); b) Glaucoma; c) Diabetic Eye Disease, and d) Cataracts. Current research has identified “oxidative stress” as a common pathological contributor in each of these ophthalmic conditions, among many forms of severe, debilitating and chronic diseases.

Age-related macular degeneration (AMD) is the leading cause of blindness in developed countries. The number of affected individuals with advanced stages of the disease in the United States alone is expected to increase nearly twofold, to approximately three million people by the year 2020. Fortunately, therapeutic options are improving. The use of antioxidant vitamins has been shown to delay disease progression at an intermediate stage, and rapid innovation in the use of antiangiogenic therapies has resulted in new clinical methods to treat the exudative phase of AMD. However, developing new treatments and prevention strategies targeting earlier stages of the disease, better known as Dry AMD (DAMD) has required a better understanding of the underlying disease mechanisms.

Findings from the Age-Related Eye Disease Study (AREDS) clearly support the hypothesis that oxidative mechanisms i.e., “oxidative stress,” play a significant role in the progression of AMD. Although several studies have shown that the intake of antioxidant-rich foods lowers the risk of AMD, others have not supported this conclusion. Cigarette smoking is a pro-oxidant stressor that significantly increases the risk of AMD, and this association is well supported in numerous, well-designed studies. The most posterior layer of the retina is the retinal pigment epithelium (RPE); which is subject to a particularly high level of oxidative stress because of locally elevated oxygen tension, high polyunsaturated lipid content (phagocytized photoreceptor outer segments), focused light exposure, direct interface with oxidative biochemicals (free radicals) generated by photoreceptor outer segment phagocytosis, and secondary photosensitizing agents (lipofuscin and APO-E) that accumulate with aging. The RPE regulates oxidative stress using neuroprotective mechanisms to detoxify reactive oxygen species through the use of antioxidant enzymes such as superoxide dismutases (e.g., cytosolic copper-zinc superoxide dismutase [CuZnSOD] or mitochondrial manganese superoxide dismutase [MnSOD]). Oxidative stress induces damage to proteins that prompts molecular chaperones such as the heat shock proteins (HSPs) and the ubiquitin-proteasome pathway to initiate self-repair mechanisms by refolding or degrading damaged proteins.

As a result of this long appreciation of the medicinal effects of foods with high antioxidant properties and the more recent elucidation of the role of the redox systems contained in the mitochondria, there has been a long felt and urgent need by the pharmaceutical and medical communities as well as the general population at large to identify compounds capable of restoring normal redox balance in diseased cells through restoration of normal redox function in the redox binding sites within the electron transport chains located in mitochondria and elsewhere in eukaryotic cells.

### SUMMARY

It is to be understood that the foregoing description is provided to merely aid the understanding of the present invention. As such, without being limited by specific examples or theory, a family of synthetic, multi-component, biologically active pharmaceutical compositions that have been shown to directly modify or inhibit mitochondrial respiration are provided herein. Thus, cellular oxidative stress may be controlled and managed thereby exerting a desired therapeutic effect on diseases that are causally associated with oxidative stress, such as ophthalmic diseases, and more specifically diseases of the retina, and even more specifically, the condition termed AMD, and most specifically the dry forms of AMD (DAMD).

In a first aspect, the disclosure provides therapeutic compositions comprising, for example, at least one or more of equilibrated peroxidic species, a stabilizing solvent; a chelating dye; and an aromatic redox compound. In certain embodiments, the equilibrated peroxidic species are derived from oxidation of unsaturated organic compounds in a liquid form or in a solution by an oxygen-containing oxidizing agent. In still additional embodiments, the equilibrated peroxidic species are stabilized in the stabilizing solvent.

In certain embodiment, the chelating dye at least one selected from the group consisting of porphyrins, rose Bengal, chlorophyllins, hemins, porphyrins, corrins, texaphrins, methylene blue, hematoxylin, eosin, ethryrosin, flavinoids, lactoflavin, anthracene dyes, hypericin, methylcholanthrene, neutral red, phthalocyanine, fluorescein, eumelanin, and pheomelanin.

In additional embodiments, the aromatic redox compound is a quinone, substituted or unsubstituted benzoquinone, or anthroquinone.

In still additional embodiments, at least one of the equilibrated peroxidic species is 5,5-Dimethyl-1,2,4-trioxolane-3-ethyl-5-methyl-1,2,4-trioxalane-3-ethan-1-ol.

In further embodiments, the stabilizing solvent is selected from the group consisting of lecithin, phosphatide, ethanol, propylene glycol, methylsulfonlmethane, polyvinylpyrrolidone, pH-buffered saline, and dimethylsulfoxide (DMSO). In a preferred embodiment, the stabilizing solvent is an anhydrous dimethylsulfoxide (DMSO).

In an embodiment of a composition as described herein, the chelating dye and the aromatic redox compound are included in an equal molar amount from about 0.001 to about 0. moles per liter of the composition and the equilibrated peroxidic species stabilized in the stabilization solvent is included in an amount from about 0.5 to about 1.0 moles per liter of the composition.

In an additional aspect, the disclosure provides a method of treating a disease, comprising administering an effective amount of a pharmaceutical composition as described herein to a subject in need thereof. In certain embodiments, the disease is associated with oxidative stresses. In certain additional embodiments, the disease is an ophthalmic disease or Age Related Macular Degeneration.

In any of the methods described herein, the pharmaceutical composition is administered intravenously.

In certain embodiments, the subject to be treated is a human.

In an additional aspect, the disclosure provides methods of synthesis of the equilibrated peroxidic species as described herein, comprising, oxidizing unsaturated hydrocarbons by ozonoylsis.

Where applicable or not specifically disclaimed, any one of the embodiments described herein are contemplated to be able to combine with any other one or more embodiments, even though the embodiments are described under different aspects of the invention.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention. The publications and other materials used herein to illuminate the background of the invention, and in particular cases, to provide additional details respecting the practice, are incorporated by reference, and for convenience are listed in the appended bibliography.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIG. 1 shows exemplary REDOXAGEN engine activities of A-F reactions.

FIG. 2 is a diagram illustrating relative energies AE of reacting systems along the reaction coordinate of the separate reactants (R), reactant complex (REDOXAGENS), transition state (TS), product complex (PC) and separate products (P) calculated at the B3LYP/6-31+G* level. The reactant complex and product complex were derived with the IRC-B3LYP/6-31+G* calculations and were not fully optimized in separate calculations. Hence the relative energies shown are not ZPVE corrected.

FIG. 3 shows results of First Seven Patients in the Observational Case Study of RC-1α in Dry AMD

FIG. 4 shows raw data from Observational Case Study of RC-1α in DMAD

FIG. 5 shows results from Proof-Of-Concept Study

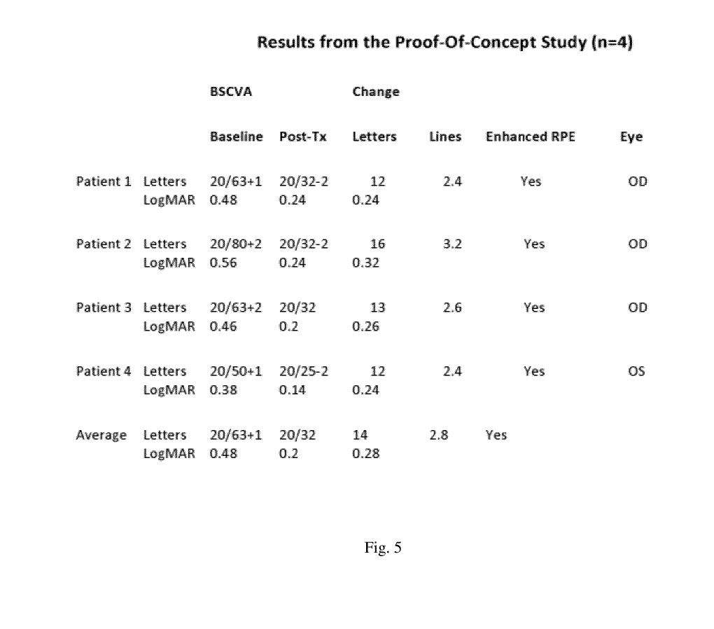

FIG. 6 shows an example OCT result from Proof-Of-Concept Study

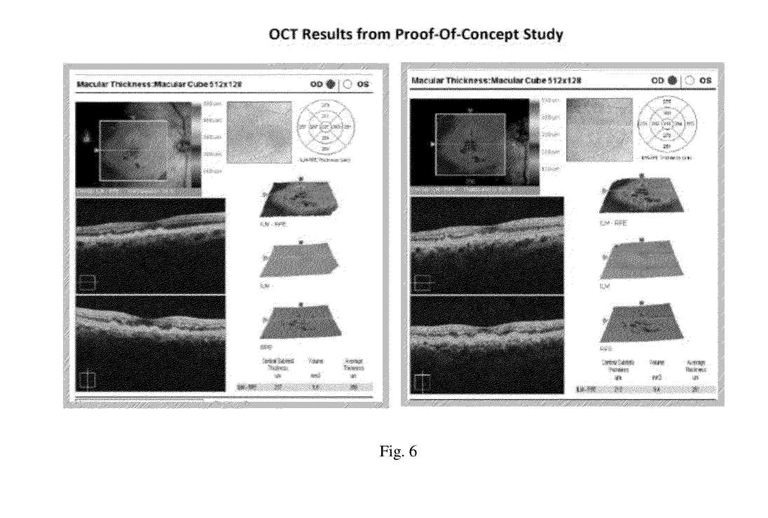

FIG. 7 shows an exemplary peroxidic species: 5,5-Dimethyl-1,2,4-trioxolane-3-ethyl-5-methyl-1,2,4-trioxalane-3-ethan-1-ol.

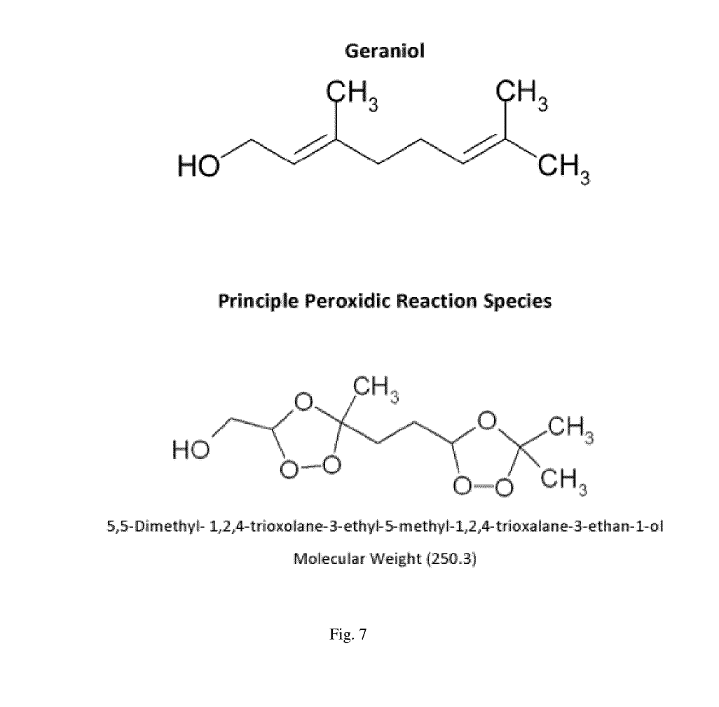

FIG. 8 shows 3-D rendering of: 5,5-Dimethyl-1,2,4-trioxolane-3-ethyl-5-methyl-1,2,4-trioxalane-3-ethan-1-ol (molecular weight 250.3).

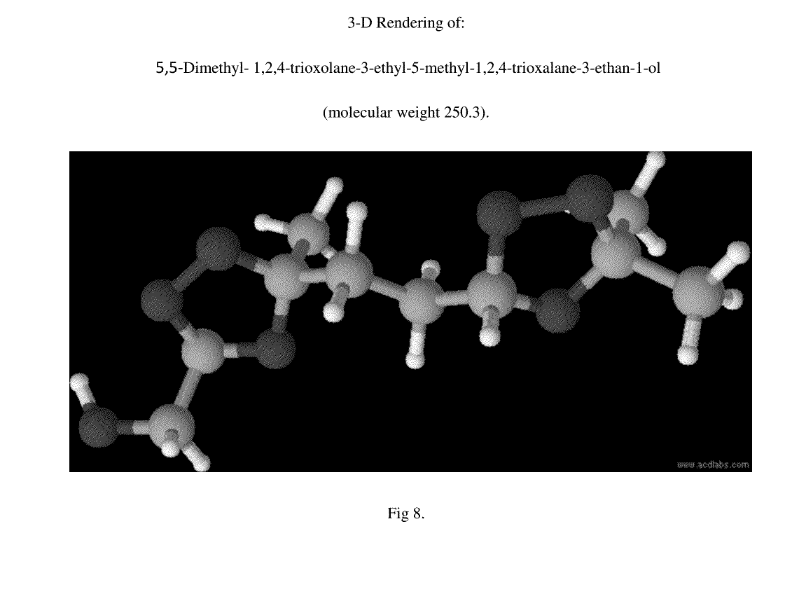

### DETAILED DESCRIPTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The term “about” or “approximately” means an acceptable error for a particular value as determined by one of ordinary skill in the art, which depends in part on how the value is measured or determined.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of’ and “consisting essentially of” shall be closed or semi-closed transitional phrases, respectively, as set forth in the 10 United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects of the present invention, one or more of the present compounds described herein, are coadministered, e.g., in a single composition. In certain additional aspects, the compositions are administered in combination with at least one additional bioactive agent. In particularly preferred aspects of the invention, the co-administration of compounds results in synergistic activity and/or therapy.

The term “treat,” “treating,” and “treatment” as used herein includes any treatment of a condition or disease in an animal, particularly a mammal, more particularly a human, and can include: (i) inhibiting the disease or condition, i.e. arresting its development; relieving at least one symptom of the disease or condition, i.e. causing regression of the condition; (ii) ameliorating or relieving the conditions caused by the disease, i.e. one or more symptoms of the disease; or (iii) eradicating the cause(s) of the disorder, disease or condition itself. The term “prevent” or “preventing” is used to refer to a prophylactic treatment of a disease or condition; i.e., preventing it from occurring or delaying its onset in a subject which may be predisposed to the disease but has not yet been diagnosed as having it.

The term “effective” is used to describe an amount of a compound, composition or component which, when used within the context of its intended use, effects an intended result.

The term “effective amount,” “therapeutically effective amount” or “pharmaceutically effective amount” refers to that amount which is sufficient to effect inhibition, treatment or prevention of a disorder, disease or pathological condition, as defined herein, when administered to a subject in need of such treatment; i.e., the therapeutically or pharmaceutically effective dose. The therapeutically effective amount will vary depending on the subject and disease state being treated, the severity of the affliction, the composition used, and the manner or route of administration, and may be determined routinely by one of ordinary skill in the art. In certain instances, the effective amount may also vary depending upon the co-administration or concurrent administration of another therapeutic, and other factors what those skilled in the art will recognize. Generally, an effective amount is between 0.01 mg/kg and 5000 mg/kg body weight/day of active ingredient. In addition, effective amounts of the compositions of the invention encompass those amounts utilized in the examples to facilitate the intended or desired biological effect.

In certain aspects, unless the context indicates otherwise, the term “pharmaceutical formulation” or “pharmaceutical composition” in this disclosure refers to a synthetic biologically active multi-component medicament that is comprised of at least one stabilized peroxidic species juxtaposed with a porphyrin dye, and an aromatic redox molecule; all of which are maintained in solution by a penetrating stabilizing solvent.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

In certain aspects, the present disclosure provides formulation, synthesis, methods, delivery and use of therapeutic multi-component pharmaceutical compositions comprising at least one desirable peroxidic species; a chelating dye; and an aromatic redox compound.

In one aspect, disclosed herein are pharmaceutical compositions of REDOXAGENS; methods of preparation; and use. Also disclosed are mechanisms of action by which these compositions achieve their therapeutic effect through the restoration of redox balance and normalization critical cellular pathways via promotion of changes in the genes of an organism thus inducing therapeutic modification of cell signaling.

The formulations contain, in various combinations and ratios, peroxidic species or reaction products resulting from juxtaposition of certain moieties including a high energy molecule derived from the oxidation of unsaturated bonds in various chemical entities such as alkenes, such as those in terpenes or monoterpenes (cyclic or acyclic), for example, geraniol, limonene (d- or l-), carveol , pinene, myrcyne, perillyl alcohol, perillic acid, cis-dihydroperillic acid, trans-dihydroperillic acid, methyl esters of perillic acid, methyl esters of dihydroperillic acid, limonene-2-diol, uroterpenol and combinations thereof, by an oxygen-containing oxidizing agent, such as ozone; along with a penetrating solvent, such as dimethyl sulfoxide (DMSO); in the presence of a dye containing a chelated metal, such as ferriprotoporphyrin or non-metal containing dyes such as hematoporphyrin; and the addition of an aromatic redox compound such as a quinone. By interchanging the specific porphyrin and quinone components and or the oxidation state of unsaturated organic compounds, these novel formulations may be useful in the treatment of a wide range of human and veterinary disease indications through direct and or indirect action on the redox binding sites, such as those co-located in mitochondria, and the intrinsic redox pathways contained therein, or downstream metabolic targets regulated by the redox state of the cell.

In particular, any of the embodiments described herein, the peroxidic species may be 5,5-dimethyl- 1,2,4-trioxolane-3-ethyl-5-methyl-1,2,4-trioxalane-3-ethan-1-ol, and or other reaction products derived from the oxidation of unsaturated organic compounds in a liquid form or in a solution, by an oxygen-containing oxidizing agent juxtaposed with a stabilizing solvent.

In another aspect, the disclosure provides a composition comprising a peroxidic species formed by ozonoylsis of an unsaturated alkene, a penetrating stabilizing solvent, a porphyrin or a metalloporphyrin, and a quinone. In certain embodiments, the composition may be formed in liquid at room temperature and ambient atmospheric pressure. The compositions as described herein can be delivered through numerous routes of administration to a patient including, intravenously, via inhalation, topically, via injections, insufflation, Iontophoresis and numerous other means of liquid formula delivery well known to those skilled in the art. The intravenous delivery method can be used to effectively treat the naive and pharmacostable dry forms of Age Related Macular Degeneration.

In another aspect, the disclosure provides a method of synthesis for improved ozonation reactions to achieve a much higher degree of control over the molecular oxygen substitution and saturation of a monoterpene's (e.g., geraniol) double bonds that is substantially above the 50% limit as disclosed in the prior art, and more preferably approaching 60, 70, 80, 90 or 100% saturation; thus providing a significantly higher concentration of the monomeric peroxidic species, for example, 5,5-dimethyl-1,2,4-trioxolane-3-ethyl-5-methyl-1,2,4-trioxalane-3-ethan-1-ol (molecular weight 250.3).

In addition, the production of high molecular weight peroxidic species in the post-reaction mixture may be reduced such that smaller, low molecular weight peroxidic species can be present in significantly higher concentrations thereby exerting a significant, consistent and measurable therapeutic effect on the RPE layer by moving across the blood brain bather to stimulate the regeneration of the RPE layer of a diseased eye and promote a durable, sustainable and significantly improved vision.

Moreover, the disclosure also provides improved formulations where Hemin is used instead of Hematoporphyrin, and Methyl-para-benzoquinone is used instead of methyl-naphthoquinone, with the elimination of Rose Bengal from the most preferred embodiment of the pharmaceutical formulation all together.

Further, the disclosure provides an improved clinical protocol comprising administering an effective amount of RC-1α, e.g., 1 ml of RC-1α, by slow IV infusion twice monthly, whereby such dosing schedule enhances the pharmacokinetics of the delivery of the pharmaceutical formulation across the blood brain bather to the tissues in the posterior retina, specifically the RPE layer of patients with DAMD in sufficient amounts such as to provide the desired therapeutic effect of neuroregeneration of the RPE layer causing a durable improvement in vision.

As such, the disclosure represents a significant breakthrough in achieving neuroregeneration in a tissue previously believed to be incapable of undergoing such self-repair. As such, the present disclosure addresses the long felt needs of the medical community and patients alike for a safe and effective pharmaceutical treatment for the treatment, prevention and management of the dry forms of Age Related Macular Degeneration and the opportunity to provide an inexpensive and easily deliverable pharmaceutical formulation that safely provides a durable restoration of vision by regenerating the architecture of the posterior retina suffering from pathological conditions.

The term “Age Related Macular Degeneration (AMD)” can refer to a medical condition which usually affects older adults and results in a loss of vision in the center of the visual field (the macula) due in part to degeneration of Retinal Pigment Epithelial layer of the retina. AMD occurs in three forms, Dry Age Related Macular Degeneration (DAMD) and Wet Age Related Macular Degeneration (WAMD), and Pharmacostable Dry AMD.

The term “Dry Age Related Macular Degeneration (DAMD)” or the non-exudative form, can refer to an early stage of the disease that refers to the form of AMD where there is a lack of neovascularization and or exudate but may be characterized by acellular debris termed Drusen which is an accumulation of electron dense lipofuscin situated between the retina and choroid and a slow progression of vision loss.

The term “Wet Age Related Macular Degeneration (WAMD)”can refer to an advanced stage of AMD typically associated with a sudden onset of significant vision loss that characteristically exhibits neovascular changes and angiogenesis originating from the choroid producing an irritating exudate leading to retinal fibrosis and scarring.

The term “Pharmacostable Thy Age Related Macular Degeneration (PSAMD)” can refer to the stage of AMD that exists after the successful administration of anti-neovascular medications that cause the retraction of neovascular ingrowth thus returning the retina to a pharmacologically induced state of prolonged Thy AMD and where vision loss is once again only slowly progressive.

The term “peroxidic species” refers to the set of biologically active, equilibrated reaction products and byproducts formed in a solution when an unsaturated hydrocarbon is oxidized, more specifically when the unsaturated hydrocarbon is an alkene, even more specifically when the alkene is a monoterpene, and most specifically when the monoterpene is geraniol that is oxidized using an ozonoylsis means and method.

The term “equilibrated peroxidic species” refers to a peroxidic species produce after the ozonoylsis of a monoterpenoid, e.g., geraniol, which may produce a number of species in equilibrium.

The term “BSCVA” refers to Best Spectacle-Corrected Visual Acuity measured using the standard ETDRS (Early Treatment Diabetic Retinopathy Study) eye chart.

The Term LogMAR refers to the measurement on the ETDRS chart defined by the ‘log of the mean angle of refraction.’

The term “subject” can refer to a cell or organism, e.g., a mammal such as a human.

In certain aspects, the present invention relates to compositions of multi-component systems. In an embodiment, the composition comprises: at least one equilibrated peroxidic species or reaction product derived from the oxidation of unsaturated organic compounds in a liquid form or in a solution by an oxygen-containing oxidizing agent, solubilized with a stabilizing solvent, in combination with a chelating dye, and an aromatic redox compound. In an exemplary embodiment of the present invention, the composition consists essentially of: the peroxidic products formed by ozonoylsis of an unsaturated alcohol; a penetrating stabilizing solvent; a porphyrin or a metalloporphyrin, and a quinone, which are collectively referred as a “REDOXAGEN” of this new therapeutic class of compounds.

Typically, working hypotheses for the composition relate to a multi-component redox modifying engine, rather than a traditional drug, which is an enzyme or receptor inhibitor. In an exemplary embodiment, the three components of the Redoxagen engine may be a porphyrin or a metalloporphyrin (a free radical generator), a quinone (an electron recycler), a polyacyloxane (a biological fuel), all solubilized in an anhydrous DMSO (a penetrant and oxane stabilizer) to stabilize the multi-component species. The porphyrin and the quinone may be included in approximately equal molar amounts 0.1 to 0.001 moles per liter, preferably 0.001 moles per liter. The equilibrated peroxidic species with its principal component being 5,5-Dimethyl-1,2,4-trioxolane-3-ethyl-5-methyl-1,2,4-trioxalane-3-ethan-1-ol (molecular weight 250.3) should be included to approximately 0.5 to 1.0 moles per liter, most preferably being 0.9 moles per liter.

In another aspect, provided herein is a pharmaceutically effective formulation which comprises an equilibrated peroxidic species derived by oxidation of unsaturated hydrocarbons, formulated with an aromatic redox compound, a chelated dye and a penetrating solvent.

In certain exemplary embodiments, the composition of the REDOXAGEN may include a “dark reaction” photodynamic component (porphyrin or dye) which is selectively absorbed into infected macrophage cells, and stimulates peroxidation and quinone-based electron transfer chains. The redox-recycling role of quinones, which are attached to the photodynamic component (porphyrin), may be facilitated by the presence of cellular oxygen, adenine nucleotides and ascorbate salts. The quinones may align alongside or attach to the polyacyloxane as a ring extension and may be presented to the porphyrin in the presence of an electron donor (ascorbate) and adenine nucleotide. As the polyacyloxane is metabolized, catalytic amounts of superoxide anion, NO, NOO, hydrogen peroxide and/or ozone are produced.

In certain exemplary embodiments of the invention the chelated dye may be at least one selected from the group consisting of porphyrins, Rose Bengal, chlorophyllins, hemins, porphins, corrins, texaphrins, methylene blue, hematoxylin, eosin, ethryrosin, flavinoids, lactoflavin, anthracene dyes, hypericin, methylcholanthrene, neutral red, phthalocyanine, fluorescein, eumelanin, and pheomelanin. Alternatively, the dyes may be, but not limited to, any natural or synthetic porphyrin, hematoporphyrin, chlorophyllins, rose Bengal, their respective congeners, or a mixture thereof. In particular, the dyes may be naturally occurring porphyrins, such as hematoporphyrin and hemin.

In certain embodiments, the aromatic redox compound may be a quinone. The aromatic redox compound may be, but not limited to, any substituted or unsubstituted benzoquinone, or anthroquinone such as benzoquinone, methyl-benzoquinone, naphthoquinone, and methyl-naphthoquinone. In certain exemplary embodiments, the aromatic redox compounds may be a benzoquinone, methyl-naphthoquinone or a mixture thereof.

In certain embodiments, the formulation may further include the penetrating agent to enhance delivery and stabilize the volatile peroxidic components of the formulation. The penetrating agent may be an emollient, a liquid, liposome, a micelle membrane, or a vapor. In addition, the penetrating agent may be an aqueous solution, fat, sterol, lecithin, phosphatide, ethanol, propylene glycol, methylsulfonlmethane, polyvinylpyrrolidone, pH-buffered saline, or dimethylsulfoxide “DMSO”. In particular, the penetrating solvent may be anhydrous DMSO, polyvinylpyrrolidone, or pH-buffered saline.

In certain exemplary embodiments, methods for the synthesis of an equilibrated consistent determinative mixture of equilibrated peroxidic species called “GEROXANE™” derived from the oxidation of the monoterpene geraniol. When formulated with a quinone and a porphyrin dye in anhydrous DMSO, the composition is called a REDOXAGEN®. In additional embodiments, the monoterpene at least one of (cyclic or acyclic), for example, geraniol, limonene (d- or l-), carveol , pinene, myrcyne, perillyl alcohol, perillic acid, cis-dihydroperillic acid, trans-dihydroperillic acid, methyl esters of perillic acid, methyl esters of dihydroperillic acid, limonene-2-diol, uroterpenol and combinations thereof.

The disclosure also provides compositions prepared for storage or administration, which include the compositions as described herein, including a pharmaceutically effective amount of the desired compounds, and further including a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

Redoxagen's action is selective; targeting only metabolically incompetent, abnormally proliferating, phagocytic (non-parenchymal) cells (macrophages, glia, retinal pigment epithelium, endothelium, etc.) that lack oxidative capacity and/or mitochondrial vigor (redox potential) and which are unable to eliminate pathogens or maintain homeostasis. Competent cells rejuvenate or return to normal function. Diseased, activated, neoplastic, infected, anaerobic, or polymerized macrophagic cells express aberrant surface receptors, undergo severe oxidative stress, release cytokines and chemokines, enter metabolic senescence, and ultimately undergo apoptosis. Redoxagen's action is based on a system of controlled ROS generation and up-regulation of oxidative stress. It does not simply inhibit enzymes—which is the basis of many current drugs.

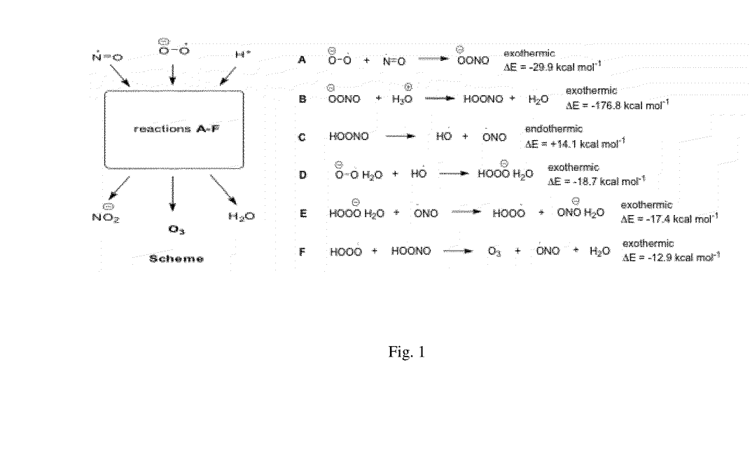

In an exemplary embodiment, the porphyrin or metalloporphyrin in a REDOXAGEN composition may allow for rapid absorption into targeted oxidatively stressed, infected or neoplastic cells, and activated or transformed macrophages, which is nearly 200 times greater than parenchymal cells such as RBCs or neuronal cells. REDOXAGEN compounds are believed to travel along select intracellular microtubules and are presented to the mitochondria or membranous redox systems as supra-molecular pro-drug assemblies. Exemplary activities of the REDOXAGEN engine are also illustrated in FIG. 1 at incompetent redox loci within the core of the mitochondrial lamellae and at select plasma membrane redox sites.

### Direct ROS/RNS Effects

Peroxide (O₂²⁻) causes a rapid depletion of mitochondrial anti-oxidant defense proteins e.g. SODs, Glutathione peroxidase (GPX), glutathione (GSH), Peroxiredoxins (PRXs), Thioredoxins (TRXs), Tocopherol and then act as a cytotoxic agent; NOX inhibits Cytochrome C Oxidase; H₂O₂ inhibits ADP phosphorylation in glycolytic and mitochondrial pathways in a dose-dependent manner as well as promotes indirect/secondary messenger changes; H₂O₂ also inhibits mitochondrial NAD levels and the activity of enzymes from Krebs cycle participants—aconitase, α-Ketoglutarate dehydrogenase (KGDH), and Succinate dehydrogenase (SDH). The presence of oxidative free radicals generates a cascade of unstable secondary intermediaries (i.e., cumulenes, carbocation radicals, carbenes, oximes and sulfenes).

Secondary metabolic/gene expression ‘ripple’ effects may include: A) Inhibition of mitochondrial function leading to Mitophagy (defective mitochondria kills itself via autophagocytosis and turns itself into a lysosome), B) Fusion (defective mitochondria fuse with a functional one to replenish stock of undamaged DNA), and C) Fission (mitochondria divide to fulfill increasing demands of self-repairing cell for ATP) (3).

Exposure to O₂²⁻ and H₂O₂ affect activity and expression of HO-1 (4), AP-1 (5), Akt (6), PKC (7) PKB (8), MAP kinases (9), catalase (10), SP1 (11), STAT3, PPARγ (12), NF-kB (13), Nrf1/2 (14), PGC-1a (15), Mn-/Mg-/Cu-SOD (10), GSH peroxidase (GPX) (9,16), GSH reductase (GSR), GSH transferase (GST) (17), glucose-6-phosphate dehydrogenase (G6PDH) (17)and hexokinase (18), which in turn effectuate a wide range of downstream responses.

The Sirtuin family of histone de-acetylases, which modulate activity of at least 34 distinct downstream targets (19), is sensitive to H₂O₂ and NAD levels that can be increased by Inhibition of KGDH in the presence of ROS/H₂O₂. (20)

Sirt1 targets p53, Aka (21), Foxo1 (22), Foxo3 (23, 24), Bax (25), Hif-1 (26), Hif-2a (27), HSF1 (28), Ku70 (29), peroxisome proliferator-activated receptor (PGC-1a) (30), b-catenin (31), E2F1 (32), Myc (33), STAT3 (34), NF-kB (35), TORC2 (36), LXR (37), FXR (38), SREBP (39), PER2 (40), CLOCK (41) and may induce energy metabolism, and promote a controlled stress response. It is also induces self-repair via secondary messengers through the activation of stem cells.

SIRT1 regulates a variety of processes that alter cell response to genotoxicity, including the detoxification of reactive oxygen species (ROS) by up-regulation of MnSOD, DNA repair mechanisms (cyclin D, GADD45, p27/Kip1) and increases the sensitivity of cells to apoptosis. This is due to the deacetylation and activation of a transcription factor FOXO3a. SIRT1 regulates the ability of FOXO3a to induce cell cycle arrest; and high SIRT1 activity promotes cell survival suggesting SIRT1 tips FOXO-dependent response away from cell death and towards stress resistance.

SIRT1 regulates both of the two types of known p53-mediated apoptotic pathways (42). p53 transcription-dependent apoptosis requires the expression of apoptosis-related target genes including BAX, PUMA, and NOXA. p53 transcription-independent apoptosis is initiated by the release of cytochrome C from the mitochondrial intra-membrane by a direct interaction between mitochondrial p53 and antiapoptotic BCL (B Cell Lymphoma, members of which bear at least one of four conserved Bcl-2 homology domains but have the opposite effect on the apoptosis process) proteins (42). In addition, Sirt2 targets Tubulin, H4, Foxo3a and may also affect cell cycle (43). Sirt3 deacetylates and maintains the normal functions of various mitochondrial proteins involved in fatty acid oxidation, ketogenesis, oxidative phosphorylation, antioxidant defense, and amino acid metabolism: mitochondrial respiratory complex I, AceCS2 (44), LCAD (45), HMG-CoA synthase 2 (45), IDH2 (46), SODs (47) and may affect ATP production, anti-oxidative stress, and thermogenesis (48). Sirt4 targets GDH and may induce Insulin secretion, and fatty acid oxidation (49). Sirt5 targets CPS1 and may affect the urea cycle (50). Sirt6 targets H3K9, H3K56, CtIP, SIRT6 and may affect DNA repair, metabolism, and inflammation (51). Sirt7 targets p53 and may affect rDNA transcription (52).

All of these mechanisms act in synergy to effectuate metabolic housekeeping, depolymerize lipids and other macromolecules (e.g., dolichols, drusen, prenylated proteins, and amyloid), or induce apoptosis in terminally diseased cells, which are unable to sufficiently neutralize the initial burst of transient oxidative stress. Collectively these actions result in the restoration of a healthy state within the cell.

### AMD and Oxidative Stress

Age-related Macular Degeneration (AMD) is believed to be a complex, multifactorial disease that appears to implicate the influences of both genetic and environmental elements and whose precise etiology remains incompletely understood. Animal models of the disease do not exist, and cell culture models typically used to investigate the role of oxidative stress cannot replicate the true biochemical mechanisms of the human condition in this disease.

In Dry AMD, it is believed that a mosaic of macular RPE cells have either inherited or develop non-lethal genetic defects (most likely located in mitochondrial DNA) which, when amplified over successive mitotic mitochondrial divisions over time (mitogenesis), combine with one or more sustained exogenous insults (i.e., chronic cadmium exposure [smoking], elevated cholesterol biosynthesis, sub-clinical hypoxia, focused UV light exposure, etc.) to impart damage to: a) cytochrome biosynthesis, and/or b) membrane-related structures (the location of oxidoreductase enzymes & cytochrome cascades) to reduce the ability of these damaged RPE cells to perform their required functions.

This slowly progressive functional degradation in RPE cells up-regulates stress responses in the mitochondria and plasma membrane redox system (PMRS) initially, then in adjacent RPE cells in an attempt to restore tissue-wide functional capacity (i.e., light processing for vision). If self-repair mechanisms are unable to reverse the incremental damage, at some physiologic threshold, the redox dysfunction begins to drive the overwhelmed cell into senescence, leading to their progressive inability to metabolize (completely oxidize) shed photoreceptor outer segments. Over time, the RPE cells become engorged, and their cell membranes begin to loose integrity; allowing their contents (i.e., A2E, terpenes, olefins, etc.) to leak into the extracellular matrix where they become trapped at the Bruch's membrane interface as an electron dense acellular lipoproteaceous matrix called “Drusen” and invoke both cellular and humoral inflammatory cascade responses.

This response consists of the release of chemokines and cytokines that stimulate complement activation and macrophage infiltration in an abortive attempt to digest these irritative (electron dense) extracellular residues ultimately leading the formation of soft Drusen.

Simultaneous releases of IL-6 and TNF stimulate the production of VEGF and other neovascularization factors in a final rescue attempt to improve molecular oxygen concentrations and nutrient delivery (oxidative capacity) and increase blood flow and choriocapillary perfusion to reduce the inflammatory stimulus. Unfortunately, the new vessels have weak tight junctions and incompetent endothelia. They leak and bleed until fibrogenesis is well-established and the local retinal photo-architecture becomes, at some point, irreversibly damaged.

Dry AMD is thus promoted by a progressive disruption of redox cellular function at the RPE/Photoreceptor interface, likely from a multitude of unrelated yet interdependent causes (genetics, decreased blood flow, fatty diet, environmental insults, etc.). This disruption initiates a self-perpetuating redox dysregulation at the level of the cytochrome cascade binding sites (electron transport chain [ETC]) in mitochondria and along the PMRS microtubule network that are propagated with clonal expansion of the daughter mitochondria in rods, cones and RPE cells

Utilizing an up-regulating charge coupling scheme mimicking native cytochrome functions, the pharmaceutical preparation of the present invention works to normalize ETC bioenergetics within the mosaic population of senescent retinal cells through the controlled upregulation of reactive oxygen species (ROS) and reactive oxygen intermediates (ROI) generation. The enhanced redox signaling promotes mitochondrial DNA to initiate Sirtuin (Sir 1-Sir 7) release that drives a series of synergistic cell repair mechanisms. This repair process has been documented in human clinical studies in the form of regeneration of the RPE layer architecture, enhanced RPE cellular metabolic activity (representative of significant mitogenesis activity), leading to a clinically relevant improvement in vision as documented herein. Restoration of vision necessarily demonstrates that this repair process is occurring in an organized fashion at the tissue, cellular as well as the intracellular level of morphology and physiology.

This disclosure includes the clinical findings that provide the evidence of the activity of the pharmaceutical preparation in this regard. Further disclosures will document the specific redox activity and mechanisms involved in modifying the oxidative stress response in order to promote improvement in vision in patients with DAMD.

### Method of Using Peroxidic Formulation

The present invention also provides improved vision to patients with eye diseases by utilizing the pharmaceutical composition to regenerate diseased ocular tissues. Therefore, it is a first primary object of the present invention to overcome the limitations and failings of the prior art by providing improved therapeutic compositions as described herein and methods of use, derived from an improved formulation and synthesis method, that: a) maximizes the generation of a desirable equilibrated peroxidic species, most specifically principally 5,5-Dimethyl-1,2,4-trioxolane-3-ethyl-5-methyl-1,2,4-trioxalane-3-ethan-1-ol (molecular weight 250.3); while at the same time, b) limits the generation of undesirable reaction species in certain ozonated mixtures. This synthesis is pursued in combination with an improved dosing and delivery protocol method, in order to provide maximum delivery of the therapeutic pharmaceutical compositions to the target tissues, to directly upregulate a specifically desired therapeutic effect within the target tissues, more specifically to neurologic tissues, even more specifically to tissues located behind the blood brain bather, even more specifically to tissues in the retina, and more specifically to the Retinal Pigment Epithelium (RPE) layer of the posterior retina of patients with AMD, and most specifically in patients with one of the dry forms of AMD, that will cause regeneration of the RPE layer in a diseased eye, leading to improved vision for a patient.

In one aspect, provided herein is a method of using the pharmaceutical formulations of an equilibrated peroxidic species formulated with methyl-para-benzoquinone and hemin in anhydrous DMSO to stimulate growth and differentiation of Retinal Pigment Epithelial cells (RPE) or Retinal Pigment Epithelial progenitor cells of the macula for the purpose of partially or completely restoring normal tissue architecture and physiologic function of the macular RPE layer to improve vision.

In another aspect, provided herein is a method of treating, preventing, or managing any disease of the eye whose cause, progression, or maintenance is influenced by a redox imbalance within a diseased cell or within the diseased cells' microenvironment by administration of the pharmaceutical formulations described herein.

In an exemplary embodiment, provided is a method of treating, preventing, or managing Dry Forms of Age Related Macular Degeneration by administering therapeutically effective amounts of equilibrated peroxidic species to the subject. Particularly, the equilibrated peroxidic species may be obtained resulting from oxidation reactions formulated with a penetrating stabilizing solvent, a porphyrin, and an aromatic redox compound.

In certain embodiments, provided herein is administration of pharmaceutical compositions disclosed herein via a variety of delivery routes including, but not limited to, intravenous; intraocular; intrathecal; transmucosal via nasal, rectal, vaginal, sublingual, pulmonary or buccal mucosal membranes; ophthalmic drops, or transdermal delivery via penetrating solvents or pars plana Iontophoresis

In certain embodiments, the pharmaceutical composition may be administered once, multiple times over a period of time, or indefinitely over the patient's lifetime.

In certain embodiments, the specific route of delivery may be determined according to the disease indication, multiplicity of treatment, duration of treatment and desired pharmacokinetics.

In certain embodiments, the subject is a non-human mammal. In other certain embodiments, the mammal is a human.

In certain embodiments, the method provided herein encompasses treating a patient regardless of patient's age, although some conditions, disease, or disorders are more common in certain age groups. In certain embodiments, the patient is male. In certain embodiments the patient is female.

In certain exemplary embodiments, the patient's age may range from about 10 to 90 years of age, 20 to about 90 years of age, from about 30 to about 90 years of age, from about 40 to about 90 years of age, from about 50 to about 90 years of age, or particularly from about 55 to about 85 years of age.

### Synthesis

The present invention also provides an improved synthesis method. As is well known to those skilled in the art, unlike straightforward chemical reactions that generate a single end-product, ozonoylsis reactions of alkenes, and of monoterpene alcohols in particular, and most specifically of geraniol, have historically generated a wide variety of peroxidic species, congeners and other byproducts in the reaction mixture based upon numerous factors. Oxidic, epoxidic and peroxidic monomers, dimers, trimers, tetramers, and numerous other oligopolymeric molecules are formed when these reactions occur in an uncontrolled fashion.

Accordingly, in one aspect, the present invention to provide an improved synthesis method that provides an improved composition of matter of a biologically active therapeutic pharmaceutical composition that: a) maximizes the generation of desirable equilibrated peroxidic species, most specifically and principally 5,5-Dimethyl-1,2,4-trioxolane-3-ethyl-5-methyl-1,2,4-trioxalane-3-ethan-1-ol (molecular weight 250.3); while at the same time, b) limits the generation of undesirable reaction species in certain ozonated mixtures, especially high molecular weight species. These requirements exist in order to maximize a direct therapeutic effect to target tissues, more specifically neurologic tissues, even more specifically tissues behind the blood brain bather, even more specifically tissues in the retina, and most specifically the Retinal Pigment Epithelium (RPE) layer of the posterior retina, with the desired therapeutic effect being regeneration of the RPE layer in a diseased eye.

In an exemplary embodiment, anhydrous DMSO may be used in the synthesis of the equilibrated peroxidic species in order to provide greater stability for the stabilized preservation of the targeted monomeric peroxidic species, particularly, 5,5-Dimethyl-1,2,4-trioxolane-3-ethyl-5-methyl-1,2,4-trioxalane-3-ethan-1-ol, as the principal final end product of the improved synthesis method thus generating an improved composition of matter for the intended use herein.

A synthesis method for the oxidation of an unsaturated organic compound may be carried out either in a solvent or neat. In either case, the cooling of the reaction is critical in order to avoid an explosive decomposition of the equilibrated peroxidic species and the formation of other less desirable reaction products of the synthesis.

In an exemplary embodiment, the present invention utilizes ozonoylsis as the chemical reaction of choice for the oxidation of unsaturated hydrocarbons.

In a particular example, a flask fitted with a mechanical stirring device is charged with an unsaturated hydrocarbon, like an alkene, such as a monoterpene like geraniol and the apparatus is weighed. The flask is surrounded by a cooling bath (ice-water, ice-salt or other suitable cooling solution). When the contents are cooled at a temperature below 5° C., at a temperature below 0° C., or particularly at a temperature from about −70° C. to about −45° C., stirring is begun and a stream of ozone in dry oxygen is passed through the mixture. It is advantageous to disperse the ozone-oxygen mixture through a glass frit, but is not necessary for a stirred solution. Periodically the gas stream is stopped and the reaction flask is weighed or the reaction mixture is sampled and analyzed by a proton magnetic resonance (¹H NMR) means. Then as deemed necessary the gas stream may be re-started in order to achieve the desired degree of reactivity and reaction efficiency of equilibrated peroxidic species production. Once the mass of the reaction flask shows sufficient weight gain, or once the proton magnetic resonance (¹H NMR) spectrum of the reaction mixture shows the desired reduction of intensity of the olefinic proton resonances, the gas stream is stopped. The reduction of intensity of the olefinic proton resonances may be varied to modify the pharmaceutical properties of the formulated and equilibrated peroxidic species end-product for improved therapeutic benefit or reduced toxic side effects for specific disease indications.

The oxidation may be carried out as above, with or without a solvent, substituting an alkenol for the alkene does not affect the reaction kinetics in any substantive manner. In an exemplary embodiment, the oxidation may be carried out with methylene chloride.

Subsequent to the oxidation reaction, the equilibrated peroxidic species may be formulated with a variety of quinones and porphyrins to yield a biologically active pharmaceutical formulation suitable for administration via a variety routes including, but not limited to, intravenous; intraocular; intrathecal; transmucosal via nasal, rectal, vaginal, sublingual, pulmonary or buccal mucosal membranes; or transdermal delivery via penetrating solvents, ophthalmic drops, or pars plana Iontophoresis. The specific route of delivery will appropriate to the disease indication and the desired pharmacokinetics.

### EXAMPLES

### Example 1

### Oxidation or Ozonolysis of a Liquid Alkene

A general procedure for the oxidation of an unsaturated organic compound may be carried out either in a solvent or neat. In either case, the cooling of the reaction may be critical in avoiding explosive decomposition of the peroxidic products of the reaction. The following general procedure is typical for the ozonoylsis of a liquid alkene.

A 1 liter flask fitted with a magnetic stirrer is charged with the alkene and the apparatus is weighed. The flask is surrounded by a cooling bath (ice-water, ice-salt or other suitable cooling solution). When the contents are cooled below 5° C., stirring is begun and a stream of anhydrous ozone in dry oxygen is passed through the mixture. It would advantageous to disperse the ozonated-oxygen through a glass frit, but may not be necessary for a stirred solution. Periodically the reaction flask is weighed or the reaction mixture is sampled and analyzed by proton magnetic resonance (¹H NMR) or other means. The gas stream is then re-started until the desired degree of reaction is obtained.

Once the mass of the reaction flask shows sufficient weight gain, or once the proton magnetic resonance (¹H NMR) spectrum of the reaction mixture shows the desired reduction of intensity of the olefinic proton resonances, the gas stream is stopped. The reduction of intensity of the olefinic proton resonances may be varied to modify the pharmaceutical properties of the formulated product for improved therapeutic benefit or reduced toxic side effects for specific disease indications.

The ozonoylsis may be carried out as described above, with or without a non-reacting solvent, substituting an alkenol for the alkene which does not affect the reaction in any substantive manner.

Subsequent to the ozonation reaction, the ozonated products may be formulated with a variety of quinones and porphyrins to yield a multi-component pharmaceutical formulation suitable for administration via a variety routes including, but not limited to, intravenous; intraocular; intrathecal; transmucosal via nasal, rectal, vaginal, sublingual, pulmonary or buccal mucosal membranes; or using a transdermal delivery means via penetrating solvents or Iontophoresis methods. The specific route of delivery will be appropriate to the targeted disease indication and the desired pharmacokinetics required to achieve the intended therapeutic effect.

### Example 2

An anhydrous ozone/pure oxygen gas mixture (approximately 90% ozone) is sparged at 1 liter of gas per hour through a solution of alkadiene alcohol, 3,7-dimethyl-2,6-ocatdiene-1-ol (geraniol) in dichloromethane at 1 atmosphere. The temperature of the reaction is about below 5° C., below 0° C., or a range from about −70° C. to about −45° C. Small aliquots of reaction product are removed hourly and ¹H NMR of the formation of the equilibrated peroxidic species and or other reaction products are measured. Alternatively, colorimetric method may be used to determine complete reaction. For example, when the mixture turns blue, the reaction is complete The reaction is stopped when at least 50%, or preferably 100% of the available unsaturated bonds have been reacted. Subsequently, the product mixture is diluted in anhydrous dimethylsulfoxide (1:10) to yield a solution or dispersion and then, the dichloromethane is removed under vacuum.

Prior to use in a targeted biological system, the equilibrated peroxidic species is diluted in a solution of methyl-para-benzoquinone and hemin (heme arginate) to yield a final pharmaceutical composition of approximately 0.28% methyl-para-benzoquinone, 0.67% hemin, equilibrated peroxidic species 1.00%, and anhydrous DMSO 98.00% given by the designated Redoxagen name RC-1α.

### Example 3

### Preparation of the Pharmaceutical Formulation

Another preferred embodiment of the invention is a pharmaceutical formulation prepared as follows:

An anhydrous ozone/pure oxygen gas mixture (approximately 90% ozone) is sparged at 1 liter of gas per hour through a solution of alkadiene alcohol, 3,7-dimethyl-2,6-ocatdiene-1-ol (geraniol) in dichloromethane at 1 atmosphere and then the temperature of the reaction is maintained at 5° C. or below. Small aliquots of reaction product are removed hourly and measuring by ¹H NMR the formation of the equilibrated peroxidic species and or other reaction products. The reaction is stopped when at least 50% of the available unsaturated bonds have been reacted. The product mixture is diluted in anhydrous dimethylsulfoxide (1:10) to yield a solution or dispersion and subsequently dichloromethane is removed under vacuum. Prior to use in a targeted biological system, the equilibrated peroxidic species is diluted in a solution of 2-methyl 1,4 naphthoquinone sodium bisulfite and hematoporphyrin IX to yield a final pharmaceutical composition of approximately 0.28% 2-methyl 1,4 naphthoquinone sodium bisulfite, 0.67% hematoporphyrin IX, 1.00% peroxidic reaction product in 98.00% anhydrous DMSO, given by the designated Redoxagen name RC-2β.

### Example 4

Seven horses afflicted with various neoplastic diseases, including squamous cell carcinoma, were selected to evaluate the therapeutic and safety potential of RC-2β as an anticancer agent. In a consensual pilot study subjects with advanced disease were given 6 RC-2β treatments by intravenous administration over a 4 week period. Prior to treatment with RC-2β tumors were biopsied to confirm the diagnosis and stage of disease. During the 2 year post treatment all subjects were followed by clinical observation to monitor efficacy and safety. In all seven cases at the end of the 2 year follow up period clinical observations showed that all tumors had completely resolved with restoration of normal anatomy and function. Biopsies of the area where tumors had existed prior to RC-2β treatment showed no histological evidence of disease. There was no clinical evidence of toxicity during or post treatment in any subject as judged by clinical observation.

### Example 5

In a series of open label, prospective, studies involving a total 101 human volunteers, ages 55 to 85, RC-1α treatment was shown to improve visual acuity and restore the retinal pigment epithelial layer in patients suffering from category 2-3 dry age related macular degeneration (DAMD). In one series, four human volunteers were treated with ten RC-1α via intravenous infusions administered every other week over an 18 week period. These volunteers presented with a diagnosis of category 2-3 DAMD with visual acuity (ETDRS) ranging between 20/40 to 20/100, with multiple large soft drusen and no fluorescein evidence of angiogenesis. At the 6 month follow up Florescein measurements showed no evidence of angiogenesis in any of the four patients and a Mean of 2.8 lines of improvement in ETDRS visual acuity. There were no cases of disease progression or significant toxicities as determined by clinical observation, digital photography, angiography or Optical Coherence Tomography (OCT). All eight eyes in this 4-patient cohort were evaluated by OCT at baseline and at the final evaluation 6-months post baseline. All eight eyes demonstrated evidence of treatment-related morphological changes consistent with regeneration of the retinal pigment epithelial layer (RPE) on OCT examination. The observation of treatment-related regeneration of the RPE occurred in concert with the documented improvement in ETDRS visual acuity.

### Example 6

Redoxagens directly affect mitochondrial redox functions, including respiration. Pulmonary artery endothelial cells (PAEC) were seeded into multiple wells in assay plates 24 hours before RC-2β treatment. Four hours before the mitochondrial stress test, the PAEC cells were divided into 3 cohorts in replicate wells and were treated with 3 different doses of RC-2β. Just prior to induction of mitochondrial stress, the cell culture media was changed. Treatment using RC-2β showed a dose dependent reduction of mitochondrial-specific oxygen consumption.

### Example 7

Redoxagens are a class of novel proprietary multi-component drugs representing a new platform of investigational therapeutic drug candidates. In brief, the Redoxagens place abnormally proliferating, infected or activated cells into a state of transient oxidative stress, forcing them to upregulate their stress-defense mechanisms to repair cellular damage, kill a microbe, metabolize microfilament prenylated cross-linkages, or force them to undergo apoptosis. This activity is accomplished through the generative promotion of various reactive oxygen species (ROS) and reactive oxygen intermediates (ROI) via signal transduction initiated by the redox pathways influenced by the Redoxagen moiety. These species include hydrogen peroxide, singlet oxygen, superoxide ion, ozone and other redox signaling molecules.

### Example 8

### Ozonolysis

In a 1-liter reaction vessel, a mixture of anhydrous ozone/pure dry oxygen gas mixture (approximately 90% ozone) is sparged at 1 liter per hour through a solution of an alkadiene alcohol, trans-3,7-dimethyl-2,6-ocatdiene-1-ol (i.e., geraniol) in dichloromethane at 1 atmosphere in the dark. The temperature of the reaction is maintained at a temperature below 5° C., at a temperature below 0° C., or particularly at a temperature from about −70° C. to about −45° C. Small aliquots of reaction product are removed hourly whereby the formation of the equilibrated peroxidic species and or other reaction products that may be used as markers of the reaction's progress are measured by ¹H NMR, or other suitable means. The reaction is stopped when at least 50% up to 100% of the available unsaturated bonds have been reacted. The final product mixture is diluted in anhydrous dimethylsulfoxide (1:10) to yield a stabilized liquid solution or dispersion. The Dichloromethane is removed from the solution under high vacuum.

In a most preferred embodiment, prior to use in a targeted biological system, the equilibrated peroxidic species is diluted in a solution of methyl-para-benzoquinone and hemin (heme arginate) to yield a final pharmaceutical composition of approximately 0.28% methyl-para-benzoquinone, 0.67% hemin, with the equilibrated peroxidic species being about 1.00%, and anhydrous DMSO comprising about 98.00% of the formulation and designated by the Redoxagen nomenclature RC-1α, where RC means Redoxagen Composition, ‘1’ means methyl-para-benzoquinone, and a means hemin.

### Example 9

### Synthesis

In a 1-liter reaction vessel, a mixture of anhydrous ozone/pure dry oxygen gas mixture (approximately 90% ozone) is sparged at 1 liter per hour through a solution of an alkadiene alcohol, trans-3,7-dimethyl-2,6-ocatdiene-1-ol (i.e., geraniol) in dichloromethane at 1 atmosphere in the dark. The temperature of the reaction is maintained at or below 5° C., or more preferably below 0° C. Small aliquots of reaction product are removed hourly whereby the formation of the equilibrated peroxidic species and or other reaction products that may be used as markers of the reaction's progress are measured by ¹H NMR, or other suitable means. The reaction is stopped when at least 50% up to 100% of the available unsaturated bonds have been reacted. The final product mixture is diluted in anhydrous dimethylsulfoxide (1:10) to yield a stabilized liquid solution or dispersion. The Dichloromethane is removed from the solution under high vacuum.

In another exemplary embodiment, prior to use in a targeted biological system, the equilibrated peroxidic species is diluted in a solution of 2-methyl 1,4 naphthoquinone sodium bisulfite and hematoporphyrin IX to yield a final pharmaceutical composition of approximately 0.28% 2-methyl 1,4 naphthoquinone sodium bisulfite, 0.67% hematoporphyrin IX, with the equilibrated peroxidic species being about 1.00%, and anhydrous DMSO comprising about 98.00% of the formulation and designated by the Redoxagen nomenclature RC-2β, where RC means Redoxagen Composition, ‘2’ means 2-methyl 1,4 naphthoquinone sodium bisulfite, and β means hematoporphyrin IX.

The biologically active pharmaceutical compositions of the present invention disclosed herein may be administered at once, or multiple times at intervals over time. The pharmaceutical compositions may be administered via a variety routes including, but not limited to, intravenous; intraocular; intrathecal; transmucosal via nasal, rectal, vaginal, sublingual, pulmonary or buccal mucosal membranes; or transdermal delivery via penetrating solvents, ophthalmic drops or pars plana Iontophoresis. The specific route of delivery will be appropriate to the disease indication and the desired pharmacokinetics.

It is understood that the precise dosage and duration of treatment may vary with age, gender, weight, and condition of the patient being treated, and may be determined empirically using known testing protocols or by extrapolation from in vitro or in vivo testing or other diagnostic data It is further understood that for any particular individual, specific dosage regimens can be adjusted over time according to the individual need and professional judgment of the person administering or supervising the administration of the formulations.

### Example 10

A Case Series of seventy six (76) DAMD patients that were treated with RC-1α in an uncontrolled, mixed protocol, dose ranging series with safety endpoints was performed. All patients were enrolled under informed consent and treated under the exemptions provided under regulations promulgated by FDAMA97 allowing physicians to formulate, compound, alter and dispense medicants to their patients under strict guidelines and prohibitions relating to consent, claims, advertising, payments and other stipulations. This study incorporated the investigation of RC-1α principally, and was used to identify the range of doses, schedules in order to establish a treatment protocol for subsequent studies. The study showed that 1 cc of the RC-1α pharmaceutical formulation diluted up to 100 cc with sterile saline and administered every other week via intravenous infusion over 15 minutes was well tolerated by patients, without significant side effects and produced the desired clinical therapeutic objectives.

### Example 11

RC-1α was evaluated in an Observational Study of 22 patients for the treatment of naive DMAD. To qualify for enrollment, patients had to be between ages of 55 and 85, have a clinical diagnosis of DMAD; a baseline BSCVA range of between 10/40 and 20/100; have morphological evidence of multiple large soft Drusen; and no past medical history or fluorescein evidence of angiogenesis. All patients were enrolled under informed consent and treated under the exemptions provided under FDAMA97. A total of 22 patients diagnosed with Category 2-3 DAMD were enrolled with a total of 32 qualifying eyes with and average baseline BSCVA of Log MAR 0.56 (BSCVA 20/80+2). Just prior to administration, 1 cc of RC-1α was diluted in 100 cc of normal saline and administered via slow intravenous infusion using a butterfly IV set placed in the dorsal wrist. Patients received an average of approximately 8 (6 to 12) equally spaced infusions over a three to six-month period.

For evaluation of safety, study endpoints included observation and recording of Significant Drug-Related Adverse Events (SAE) and Minor Drug-Related Adverse Events (MAE); and fluorescein angiograms Efficacy endpoints were Best Corrected Visual Acuity (BCVA) using the Snellen Chart at 20 feet and patient subjective reporting.

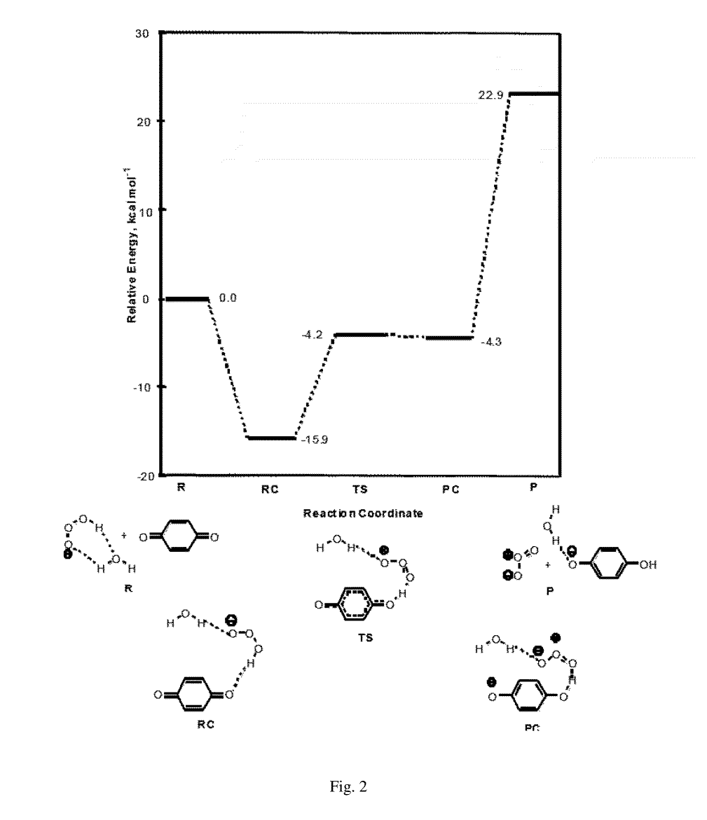

Seven of twenty two patients received independent 3rd party BCVA evaluation at both pretreatment and six months post treatment visits. This cohort showed a mean response of 2.8 lines of vision improvement from baseline BCVA to final BCVA. (FIGS. 1 and 2).

In this uncontrolled observational study 173 treatments were provided to 22 patients for an average of 7.88 treatments per patient resulting in an improvement of BSCVA line change of 3.9 lines. The raw data are summarized in FIG. 2. Twenty four (24) eyes (77%) showed improvement in BSCVA at the end of the study. No eyes (0%) worsened. Sixteen patients (73%) were able to qualify for legal driving.

No patient experienced a significant drug-related adverse event. Three minor drug-related adverse events were observed. One patient experienced a photo-reactive rash. This patient did not follow the protocol to stay out of direct sunlight for 24 hours after treatment. The rash responded to IV Vitamin E. A second patient who received too high of an initial dose experienced mild to moderate generalized joint pain which resolved spontaneously without intervention. A third patient experienced symptomatic hypoglycemia. This patient was a diabetic who had not eaten prior to treatment. Symptoms resolved upon administration of 1 liter of 5% intravenous glucose.

### Example 12

RC-1α was evaluated in a 6-month, proof of concept, randomized, prospective, double masked, eye-controlled vision study in category 2-3 DAMD patients. All patients were enrolled and treated under informed consent with Institutional Review Board sanction. Enrollment criteria were: patients had to be between the ages of 55 and 85 with a clinical diagnosis of category 2-3 DAMD; BSCVA Range of between 20/40 and 20/100; with multiple large soft Drusen; and no prior medical history or fluorescein evidence of angiogenesis. Using these criteria 4 patients were enrolled in the pilot phase of the study yielding 5 of 8 eyes qualifying for the study. Patients were evaluated at baseline, 3 months and 6 months post baseline by ETDRS BSCVA, digital photography, fluorescein angiograms and OCT imaging. At each of ten treatment sessions, patients were treated with 1 cc of RC-1α diluted to 100 cc with normal saline. Diluted RC-1α was administered by intravenous infusion as above. All 4 patients received a total of 10 evenly spaced treatments administered over an 18-week treatment period.

Safety was evaluated by monitoring for significant drug-related adverse events (SAE), minor drug-related adverse events, protocol digital photography, fluorescein angiograms, and optical coherence tomography (OCT) imaging.

Efficacy was measured by pre and post ETDRS BSCVA at 20 feet, protocol digital photography, fluorescein angiograms, OCT imaging and patient subjective reporting.

No significant adverse events were observed over the course of the study. One patient was observed to experience an asymptomatic 20 mm Hg increase in systolic blood pressure during their initial treatment that resolved with P.O. Captopril. It was undetermined whether treatment anxiety or experimental drug effects were the cause. The increase in blood pressure did not occur in the 9 subsequent treatments.

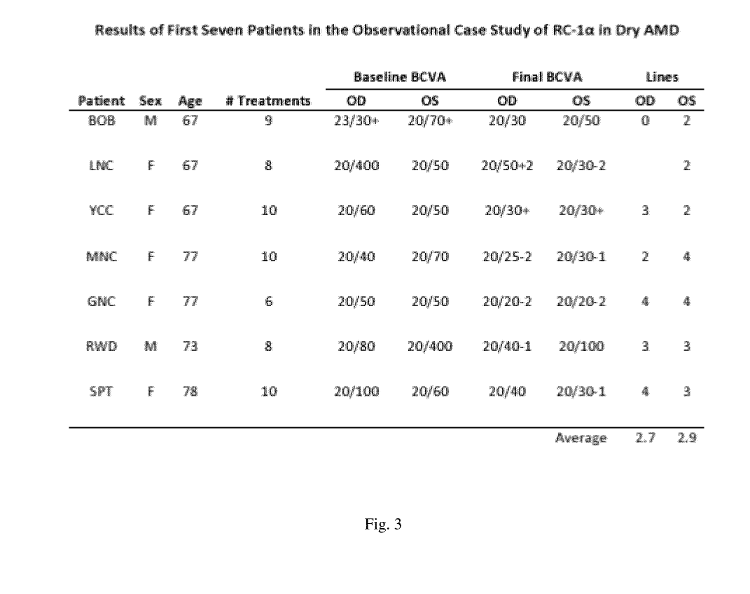

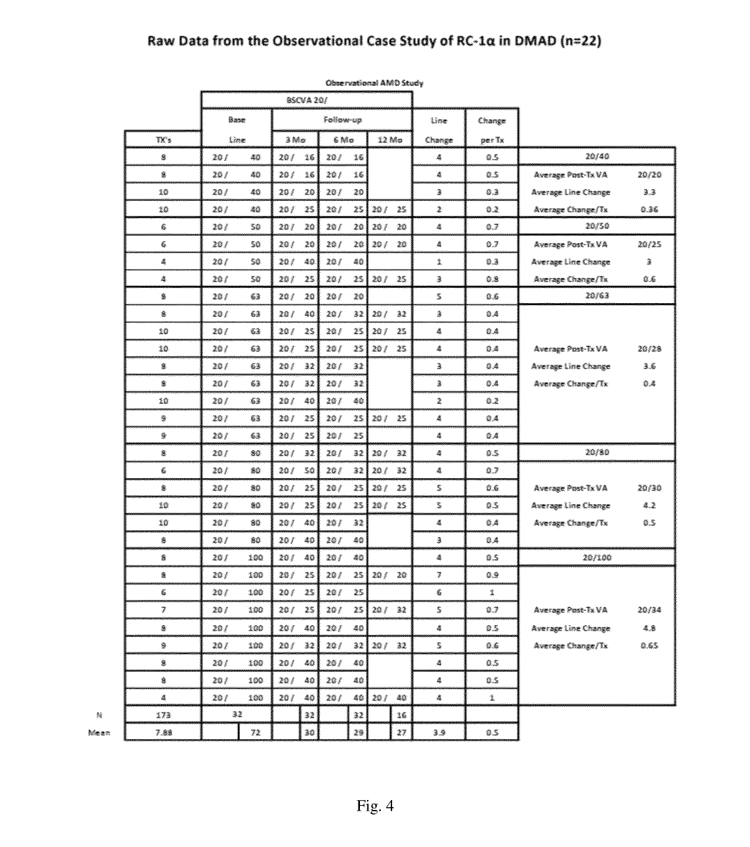

All (4/4) patients experienced improvement of visual acuity with a study average of 2.8 lines of vision improvement as measured by protocol ETDRS BSCVA methods in the 4 study eyes. (FIG. 3) All eyes (8/8) experienced a full or at least a partial restoration of the retinal pigment epithelial (RPE) layer as determined by OCT reflectance measurements (FIG. 4). To ensure the accuracy and validity of the OCT interpretations, an independent retinologist who was a widely recognized expert in OCT evaluations was recruited to interpret the OCT results in a blinded fashion; and confirmed the OCT interpretations made by the study's clinicians. No eye lost visual acuity over the course of the study. No evidence of angiogenesis was observed in any eye during the course of the study.

Given the relatively low dose of the pharmaceutical preparation RC-1alpha, and its intravenous route of administration, RC-1α must cross the blood-brain bather with very high order kinetics to exert these highly significant clinical, morphological and physiological effects on vision and the RPE layer; which lies on the opposite side of Bruch's Membrane from the choroidal (sole) blood supply.

Disclosed in this Patent Application are formulations, methods of synthesis, use and clinical results of biologically active multi-component pharmaceutical agents containing at least one equilibrated peroxidic species derived from ozonated unsaturated organic compounds. The formulations are provided for the safe and effective treatment of the naive Dry form of Age Related Macular Degeneration in humans resulting in full or partial restoration of the architecture of the Retinal Pigment Epithelium layer and the concomitant improvement in the cellular physiology of the neuroretina as evidenced by the improved visual acuity in 5/5 qualifying eyes.

The examples set forth above are provided to give those of ordinary skill in the art with a complete disclosure and description of how to make and use the claimed embodiments, and are not intended to limit the scope of what is disclosed herein. Modifications that are obvious to persons of skill are intended to be within the scope of the disclosure.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

What is claimed:

1. A pharmaceutical composition, comprising:
at least one or more of equilibrated peroxidic species;
a stabilizing solvent;
a chelating dye; and
an aromatic redox compound.

2. The pharmaceutical composition of claim 1, wherein the equilibrated peroxidic species are derived from oxidation of unsaturated organic compounds in a liquid form or in a solution by an oxygen-containing oxidizing agent.

3. The pharmaceutical composition of claim 1, wherein the equilibrated peroxidic species are stabilized in the stabilizing solvent.

4. The pharmaceutical composition of claim 1, wherein the chelating dye at least one selected from the group consisting of porphyrins, rose Bengal, chlorophyllins, hemins, porphyrins, corrins, texaphrins, methylene blue, hematoxylin, eosin, ethryrosin, flavinoids, lactoflavin, anthracene dyes, hypericine, methylcholanthrene, neutral red, phthalocyanine, fluorescein, eumelanin, and pheomelanin.

5. The pharmaceutical composition of claim 1, wherein the aromatic redox compound is a quinone, substituted or unsubstituted benzoquinone, or anthroquinone.

6. The pharmaceutical composition of claim 1, wherein at least one of the equilibrated peroxidic species is 5,5-Dimethyl-1,2,4-trioxolane-3-ethyl-5-methyl-1,2,4-trioxalane-3-ethan-1-ol.

7. The pharmaceutical composition of claim 1, wherein the stabilizing solvent is selected from the group consisting of lecithin, phosphatide, ethanol, propylene glycol, methylsulfonlmethane, polyvinylpyrrolidone, pH-buffered saline, and dimethylsulfoxide (DMSO).

8. The pharmaceutical composition of claim 6, wherein the stabilizing solvent is an anhydrous dimethylsulfoxide (DMSO).

9. The pharmaceutical composition of claim 2, wherein the chelating dye and the aromatic redox compound are included in an equal molar amount from about 0.001 to about 0. moles per liter of the composition and the equilibrated peroxidic species stabilized in the stabilization solvent is included in an amount from about 0.5 to about 1.0 moles per liter of the composition.

10. A method of treating ophthalmic disease or Age Related Macular Degeneration, comprising administering an effective amount of the pharmaceutical composition comprising at least one or more of an equilibrated peroxidic species, a stabilizing solvent, a chelating dye, and an aromatic redox compound to a subject in need thereof, wherein the composition is effective in treating or ameliorating a symptom of ophthalmic disease or Age Related Macular Degeneration.

11. (canceled)

12. (canceled)

13. The method of claim 10, wherein the effective amount of the pharmaceutical composition is administered intravenously.

14. The method of claim 10, wherein in the subject is a human.

15. A method of synthesis of the equilibrated peroxidic species in claim 1 comprising, oxidizing unsaturated hydrocarbons by ozonoylsis.

16. The method of claim 10, wherein the equilibrated peroxidic species are derived from oxidation of unsaturated organic compounds in a liquid form or in a solution by an oxygen-containing oxidizing agent.

17. The method of claim 16, wherein the equilibrated peroxidic species are stabilized in the stabilizing solvent.

18. The method of claim 10, wherein the chelating dye at least one selected from the group consisting of porphyrins, rose Bengal, chlorophyllins, hemins, porphyrins, corrins, texaphrins, methylene blue, hematoxylin, eosin, ethryrosin, flavinoids, lactoflavin, anthracene dyes, hypericine, methylcholanthrene, neutral red, phthalocyanine, fluorescein, eumelanin, and pheomelanin.

19. The method of claim 10, wherein at least one of the equilibrated peroxidic species is 5,5-Dimethyl-1,2,4-trioxolane-3-ethyl-5-methyl-1,2,4-trioxalane-3-ethan-1-ol.

20. The method of claim 17, wherein the stabilizing solvent is selected from the group consisting of lecithin, phosphatide, ethanol, propylene glycol, methylsulfonlmethane, polyvinylpyrrolidone, pH-buffered saline, dimethylsulfoxide (DMSO), and anhydrous dimethylsulfoxide (DMSO).

21. The method of claim 10, wherein the chelating dye and the aromatic redox compound are included in an equal molar amount from about 0.001 to about 0. moles per liter of the composition and the equilibrated peroxidic species stabilized in the stabilization solvent is included in an amount from about 0.5 to about 1.0 moles per liter of the composition.
